(19) United States
(12) Patent Application Publication
(10) Pub. No.: US 2024/0143697 A1
(43) Pub. Date: May 2, 2024

(54) Method For Non-Destructive Testing Of A Plurality Of Components

(71) Applicant: Electricite de France, Paris (FR)

(72) Inventors: Nicolas Paul, Montreuil (FR); Laura Couret, Levallois-Perret (FR)

(73) Assignees: Electricite de France, Paris (FR); Electricite de France, Paris (FR)

(21) Appl. No.: 18/384,671

(22) Filed: Oct. 27, 2023

(30) Foreign Application Priority Data
Oct. 28, 2022 (FR) 2211287

(51) Int. Cl. G06F 17/18; G01N 29/44

(52) U.S. Cl. CPC G06F 17/18; G01N 29/44

(57) ABSTRACT

A method for non-destructive testing of a plurality of components based on measurement signals, each of the measurement signals corresponding to a predetermined different component, including the following steps: obtaining a sequence of at least one signal portion based on each measurement signal, for each pair associating two signal portions having one and the same position in their respective sequence, a distance between these signal portions of the pair is computed, the distance being computed using dynamic time warping, for each signal portion, determining at least one statistical indicator based on the distances of pairs involving said signal portion, the statistical indicator being associated with the component of said signal portion, and for each component, comparing at least one indicator of the component to indicators of other components, and as a function of the comparison, determining the soundness or unsoundness of the component.

### TECHNICAL FIELD

The invention relates to the field of non-destructive testing of a plurality of components, in order to determine the soundness or unsoundness of the component. More precisely, the invention is applicable to the testing of similar components based on measurement signals, each of the measurement signals corresponding to a predetermined different component.

### TECHNOLOGICAL BACKGROUND

Non-destructive testing methods are based on the search for anomalies in measurement signals, liable to correspond to a defect in an unsound component. The measurement signals may be of different kinds, and for example are signals resulting from ultrasound captures, or electrical responses to stimulation such as the generation of eddy currents, the detection of radio waves, or else images or videos.

In these measurement signals, the search for anomalies can be difficult. This is because many factors are liable to alter the measurement signals, such as for example a complex geometry of the inspected component or a defect in its surface cleanliness, and the presence of an anomaly may be hidden among these alterations. Furthermore, if the detection must be done by a human operative analyzing the measurement signals, the testing can be very long and arduous, and its quality depends on the competence of this operative. These problems may become very problematic in fields where the number of components to be inspected is large and/or when it is essential to be able to detect all the defects for safety reasons.

Provision has been made for various methods allowing the search for anomalies liable to correspond to defects in measurement signals. Most of these methods are based on the comparison of values derived from the signals to references. However, each method is then specific to a particular application, and the sensitivity of the method greatly depends on the relevance of the computation of the derived values and on the definition of the reference used. These methods therefore generally require a long development time and may not be suitable for detecting unanticipated defects or for inspecting other components than those for which it was designed.

For example, the patent application FR3029288 makes provision for an inspection of vessel bottom head penetrations: for each time sample, statistical values such as the mean or the standard deviation of the values taken by the signal at this time are computed over the P available signals. These statistics are then used to construct a temporally homogenized signal, with for each time sample, a constant mean equal to zero and a constant standard deviation equal to 1. Although the application of this method to vessel bottom head penetrations gives satisfactory results, it is not as effective when applied to the inspection of other components such as baffle bolts. This is because the defects do not always give rise to increases in amplitude, but often variations in signal shape. Moreover, the signals are not exactly synchronized with one another, and local expansions/contractions/shifts may appear.

### Overview of the Invention

The invention thus makes provision for a method for non-destructive testing of a plurality of components based on measurement signals, which is simple to implement, applicable to the inspection of a large number of similar components, and makes it possible to reliably detect defects.

Provision is made for a method for non-destructive testing of a plurality of components based on measurement signals, each of the measurement signals corresponding to a predetermined different component, comprising the following steps:
obtaining a sequence of at least one signal portion based on each measurement signal,
for each pair associating two signal portions having one and the same position in their respective sequence, a distance between these signal portions of said pair is computed, said distance being computed using dynamic time warping,
for each signal portion, determining at least one statistical indicator based on the distances of pairs involving said signal portion, the statistical indicator being associated with the component of said signal portion,
for each component, comparing at least one indicator of said component to indicators of other components, and as a function of the comparison, determining the soundness or unsoundness of the component.

The dynamic time warping is here applied originally, since a statistical indicator based on the pair distances is then used to detect the signal portions distinguishable from the others.

This method is advantageously completed by the following features, taken alone or in any of their technically possible combinations:
two signal portions that follow one another in a sequence obtained from one and the same measurement signal partially overlap,
the plurality of components comprises P components, P being a natural integer greater than 10, and each signal portion is involved in P-1 pairs
a statistical indicator is a central tendency indicator, such as the mean or the median,
a statistical indicator is a dispersion indicator measuring the variability of the distances, such as the standard deviation or the variance,
at least two statistical indicators are determined, comprising a central tendency indicator and a dispersion indicator,
the comparison of at least one indicator of said component to indicators of other components comprises the determination of a difference between said statistical indicator and another nearer statistical indicator of another component,
at least two statistical indicators are determined, and the comparison of the indicator pair of one component to indicator pairs of other components comprises a statistical modelling of a probability density of the values taken by the pair of two statistical indicators of said component,
each signal portion corresponds to a measurement distance at least equivalent to a typical size of sought defect.

The invention also relates to a computer program product comprising program code instructions for executing the steps of the method according to the invention, when said program is executed on a computer. The computer program product can take the form of a non-volatile medium on which the instructions are stored, for example a memory such as a hard drive, an SSD, a flash memory, etc.

### OVERVIEW OF THE FIGURES

Other features, aims and advantages of the invention will become apparent from the following description, which is illustrative and non-limiting, and which must be read with reference to the appended drawings, wherein:

FIG. 1 is a diagram schematically showing steps for implementing a method according to a possible embodiment of the invention;

FIG. 2 shows an example of alignment by dynamic time warping between two signal portions of sound components;

FIG. 3 shows an example of alignment by dynamic time warping between a signal portion of a sound component and a signal portion of an unsound component;

FIG. 4 shows an example of a histogram of pair distances for a sound component;

FIG. 5 shows an example of a histogram of pair distances for an unsound component;

FIG. 6 shows an example of distribution of values for two statistical indicators.

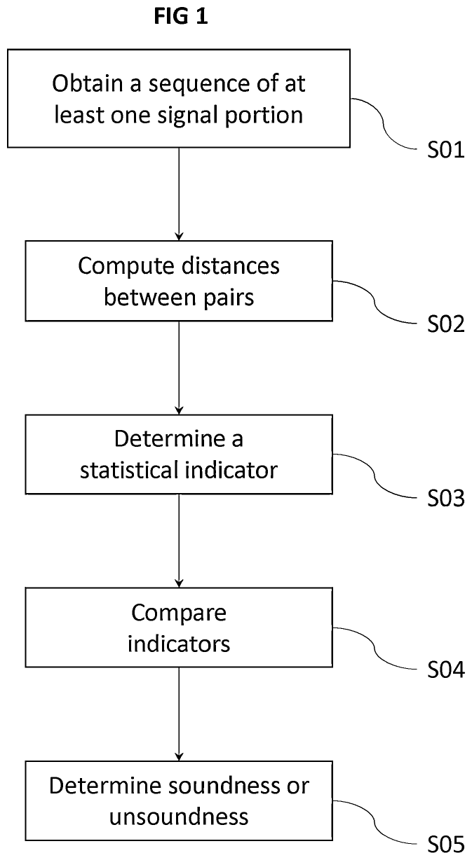

### DETAILED DESCRIPTION

The invention is applicable to the non-destructive testing of a plurality of similar components, a majority of which is considered to be sound. The term “unsound” should be understood to mean a component having a defect, for example mechanical (crack, corrosion etc.), which makes it potentially unfit to fulfil its function. For example, an attaching member such as a screw is no longer sound if cracks are detected in its body. The components can be of various types, provided that measurement signals exist, each corresponding to a predetermined different component. While the method can be implemented with a limited number of components, such as for example at least 10 components, it is more reliable and more advantageous to implement it over a large number of components. Consequently, the plurality of components preferably comprises at least 50 components, and still preferably at least 100 components.

The measuring signals may be of various kinds, as long as the measurement signals are able to exhibit variations related to the soundness or unsoundness of an inspected component, and in particular variations as a function of the presence of mechanical defects in the inspected components. For example, the measurement signals may result from ultrasound captures, or electrical responses to stimulations such as eddy currents, the detection of radio waves, or else images or videos. By way of non-limiting example, the method is described in an application to the testing of mechanical attaching components such as baffle bolts, and with measurement signals from ultrasound captures. Even in this application, the measurement signals could be different. Note that the method may comprise the capturing of the measurement signals by means of a probe or a sensor near each of the components, for example from one component to the other in succession.

The method aims to determine the soundness or unsoundness of each component. It is then possible to continue the method with a more in-depth inspection of the components considered to be unsound.

The method is implemented on a set of measurement signals, each of the measurement signals corresponding to a predetermined different component. For example, each measurement signal can be specifically captured at a respective component, typically by placing a probe near the component to be inspected: the measurement signal is then naturally associated with a particular component. In our example, an ultrasound probe is placed against a baffle bolt, then sends and receives ultrasound waves from this position against the baffle bolt. It is also possible that the initial capture comprises measurement signals of several components. This is for example the case when a probe is moved along a trajectory or successively encounters several components to be inspected. Splitting the result of the capture makes it possible to retrieve measurement signals each corresponding to a predetermined different component.

Preferably, the measurement signals are of similar sizes, in temporal and/or spatial terms, which simplifies their common manipulation in the rest of the method.

A sequence of at least one signal portion is obtained based on each measurement signal (step S1). Preferably, several signal portions forming a sequence are extracted from each measurement signal. If the measurement signal comes from the propagation of a wave, for example ultrasound, or the travel of a wave, each signal portion is a time window corresponding to a distance. Thus, each signal portion may correspond to a measurement distance, which is chosen to be equivalent to at least one typical size of sought defect, and more advantageously to approximately twice a typical size of sought defect. Typically, the sought defects are in the order of the millimeter (cracks, crazing etc.) such that each signal portion corresponds to a measurement distance between 1 mm and 4 mm. In the following example, each signal portion corresponds to a measurement distance which is a depth of approximately 2 mm.

Preferably, at least 2 signal portions are extracted from one and the same measurement signal, and preferably at least 4 signal portions are extracted from one and the same measurement signal. However, the number of signal portions does of course depend on the size of the measurement signal and on the size of the sought defects. Note that if the measurement signal is small enough, it may only be necessary to extract one signal portion, and in this case the sequence of signal portions may comprise only one signal portion. Preferably, the signal portions of two different components, having one and the same position in their respective sequence, are of comparable size, for example in terms of measurement depth. Thus, the extraction of signal portions can also serve to ensure that comparable signal portions pertain to similar areas of the inspected components, and therefore that these signal portions of different components are comparable.

Consequently, even when the measurement signals are of small sizes such that only one signal portion is extracted, the extraction can serve to at least make sure that signal portions of different measurement signals are similar (not necessarily identical) in terms of physical representativeness of the measurement (for example a measurement or penetration distance). If the measurement signals are already similar to one another, the obtainment of the sequence of at least one signal portion may simply consist in the providing of the measurement signal.

Preferably, two successive signal portions in a sequence of one and the same measurement signal partially overlap. In other words, certain data of the measurement signal are located in these two successive signal portions, or a part of a signal portion is also in another signal portion. The overlap with another signal portion is preferably between 0.1% and 25% of a signal portion, and preferably between 0.5% and 10% of a signal portion.

The signal portions form a sequence in the sense that they are ordered with respect to one another: there is a first signal portion, followed by a second signal portion, followed by a third signal portion, etc . . . . The n^{th} signal portion of the i^{th} measurement signal can be written a_{i,n}. Note that all the obtained portions of a measurement signal are not necessarily part of the sequence of the measurement portions, but that two signal portions of the sequence retain the same order as in the measurement signal: the first signal portion is in the measurement signal before the second signal portion, the third signal portion is in the measurement signal before the second signal portion, etc.

Next, for each pair associating two signal portions having one and the same position in their respective sequence, a distance between these signal portions of said pair is computed: let dtwₙ(a_{i,n}, a_{j,n}) the distance between the n^{th} portion of the i^{th} measurement signal and the n^{th} portion of the j^{th} measurement signal. As described above, the obtained portions of a measurement signal are not necessarily part of the sequence of measurement portions, and for example the sequence of measurement portions may only start from a certain extracted portion onwards, the preceding signal portions then not being used in the comparison and therefore not forming part of the measurement portion sequence. Similarly, it is possible that the signal portions of the sequence are not immediately consecutive in the measurement signal. However, it is constant that the order of the signal portions is observed: the (n+1)^{th} portion of the i^{th} signal is compared to a measurement portion of the i^{th} measurement signal which is, in the measurement signal, after the portion of the i^{th} measurement signal to which the n^{th} portion of the i^{th} measurement signal is compared. As each measurement signal is associated with a particular component, one can also define this distance dtwₙ(a_{i,j}, a_{j,n}) as the distance between the n^{th} portion of the i^{th} component and the n^{th} portion of the i^{th} component. The distance here is understood in the sense of a similarity criterion or rather one of dissimilarity (the greater the distance, the greater the dissimilarity). It is in particular a question of the distance taken into account in the dynamic time warping.

For this purpose, the distance is computed using DTW (Dynamic Time Warping), which is a method already used in other applications, making it possible to measure the similarity between two sequences which may vary. Generally, DTW is a method which searches for an optimal match between two time-domain sequences, under certain restrictions. The time-domain sequences are warped by non-linear transformation of the time-domain variable to determine a measurement of their similarity, independently of certain non-linear time transformations.

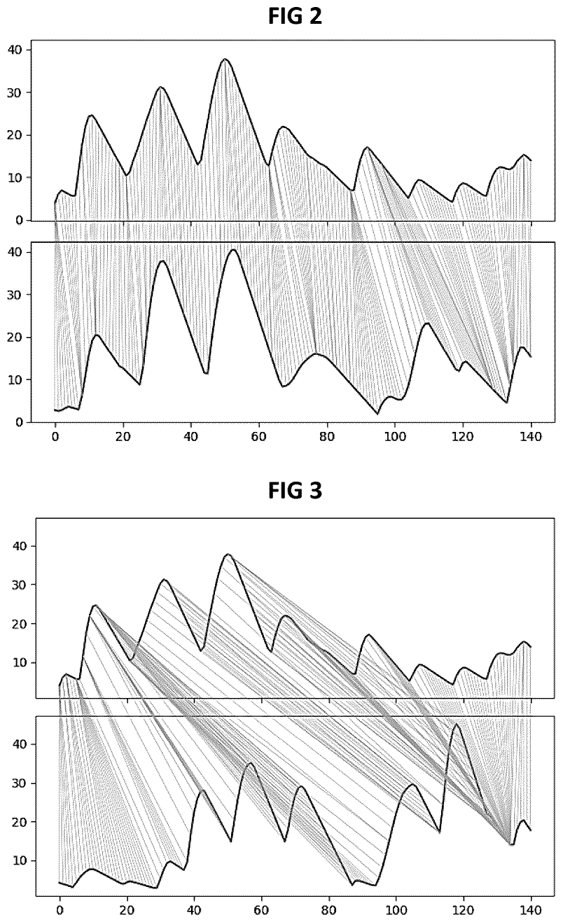

DTW defines a distance which makes it possible to measure the similarity between two portions s₁ and s₂ in a more relevant way than by comparing the signals point by point, as is for example the case with the Euclidian distance. With DTW, one point of s₁ is associated with one or more points of s₂ (and vice versa), on the basis of a minimization of a cost function. This makes the measurement particularly suitable for the analysis of time-domain sequences since it is robust to time-domain expansions, compressions and shifts between the two signal portions. FIG. 2 shows an example of alignment by time domain warping between two signal portions of sound components. The matches are illustrated by the lines connecting the points of the curves corresponding to the two signal portions. Although most of the lines are globally vertical, approaching a Euclidian distance between synchronous points, certain match lines have steep gradients, showing the effect of the transformations undergone, which, owing to the DTW, do not prevent a high degree of similarity from appearing.

FIG. 3 shows an example of alignment by time domain warping between one signal portion of a sound component and one signal portion of an unsound component. By comparison with FIG. 2, steeper gradients of the match lines can be seen, and therefore greater lengths, i.e. longer matching distances, indicating a more significant warping to be applied to match the signal portions. Furthermore, it can be seen that certain points of a signal portion are matched with points of the other signal disposed at various places on this other signal. For example, several peaks of the signal portion at the top of FIG. 2 are matched with the same point at the end of the signal portion at the bottom of FIG. 3. Even after alignment, this will result in a final distance, a dissimilarity measurement obtained by computing the deviations between the amplitudes of the points after matching, which will remain significant, since all these peaks cannot be aligned with the same point of the other signal portion.

It is however difficult to formulate hypotheses about the quality of the alignment by DTW between two signal portions of unsound components. It can happen that two signal portions of unsound components have similarities, and in this case the measurement DTW may be in the same order of magnitude as the DTW between two respective signal portions of two sound components. It can also happen that the alignment between two respective signal portions of unsound components is very complicated, and in this case the DTW measurement may be in the same order of magnitude as the DTW between a signal portion of a sound component and a signal portion of an unsound component. The preliminary step of splitting the signal into a sequence of signal portions makes it possible to have orders of magnitude of DTW that are local and comparable. Specifically, if one were to take the measurement of the DTW over the entire measurement signal, the impact of a warp at a given point in time would be too discrete to be identified.

The DTW is applied to each pair associating two signal portions having one and the same position in their respective sequence, such that if the plurality of components comprises P components, P being a natural integer greater than 10, each signal portion is involved in P-1 pairs and this would result in P-1 distances involving this signal portion and therefore a particular component. Preferably, the distances are computed for several signal portions, and preferably for all the signal portion sequences.

This approach can be expressed via a matrix Mₙ of dimension P×P with P the number of components and therefore of measurement signals. The components Mn_{i,j} of the matrix are: Mn_{i,j}, dtwₙ(a_{i,n}, a_{j,n}), with dtwₙ(a_{i,n}, a_{j,n}) the distance between the n^{th} portion of the i^{th} component and the n^{th} portion of the i^{th} component, more simply written as dtwₙ(aᵢ, aⱼ):

$${M}_{n} = ( \begin{matrix}{dtw}_{n} ( {a}_{1} , {a}_{1} ) & {dtw}_{n} ( {a}_{1} , {a}_{2} ) & … & {dtw}_{n} ( {a}_{1} , {a}_{P} ) \\ {dtw}_{n} ( {a}_{2} , {a}_{1} ) & {dtw}_{n} ( {a}_{2} , {a}_{2} ) & … & {dtw}_{n} ( {a}_{2} , {a}_{P} ) \\ ⋮ & ⋮ & ⋱ & ⋮ \\ {dtw}_{n} ( {a}_{N} , {a}_{1} ) & {dtw}_{n} ( {a}_{P} , {a}_{2} ) & … & {dtw}_{n} ( {a}_{P} , {a}_{P} )\end{matrix} )$$

This matrix Mₙ is symmetrical, with a diagonal of zeroes, and contains all the possible pair distances for one and the same position in the sequences obtained based on the measurement signals. Note that matrix notation is not indispensable, but does make it possible to organize the distances. Thus, the column j of the matrix Mₙ contains the distance dtwₙ(aⱼ, aⱼ) between aⱼ the n^{th} signal portion of the component j and itself, which has a value of 0, and the set of the distances dtwₙ(aₖ, aⱼ) between aⱼ the n^{th} signal portion of the component j and the P-1 other n^{th} portions aₖ of the other components, k≠ⱼ.

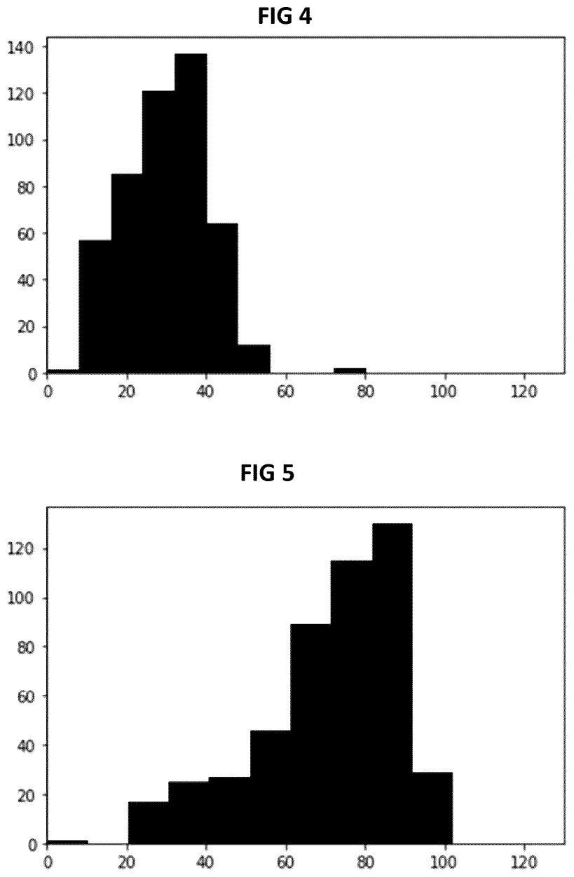

However, possessing these pair distances does not make it possible to immediately make a conclusion about the soundness or unsoundness of an component, since there is no reference with which to compare them. It is therefore preferable to adopt a statistical approach with the aim of identifying the components having signal portions, the characteristics of which deviate with respect to the others. By way of illustration, in the application of the baffle bolt inspection, FIG. 4 shows an example of a histogram of pair distances for a sound component, while FIG. 5 shows an example of a histogram of pair distances for an unsound component. On the abscissa are the pair distances for this component, and on the ordinate the number of distances in classes of distance values. Of course, the indicated values are only indicative, and dependent on many parameters such as measurement signal amplitudes. It is noted that the histogram of pair distances for an unsound component is generally translated and expanded with respect to the histogram of pair distances for a sound component. Thus, a statistical approach can make it possible to identify such deviations.

Consequently, for each signal portion, at least one statistical indicator is determined based on the pair distances involving said signal portion (step S03), the statistical indicator being associated with the component of said signal portion. Once again using matrix notation, the pair distances involving a component j, and thus involving the signal portion a j, corresponds to the column j of the matrix Mₙ.

Preferably, a statistical indicator is a central tendency indicator, such as the mean or the median, or a statistical indicator is a dispersion indicator that measures the variability of the distances, such as the standard deviation or the variance. Preferably, at least two statistical indicators are determined, comprising a central tendency indicator and a dispersion indicator. Typically, the statistical indicators are the mean μ and the standard deviation σ. Note that the statistical indicator or indicators may be determined directly based on the pair distances, or else based on a histogram such as that illustrated in FIGS. 4 and 5.

For each component, a comparison is then made of at least one indicator of said component to indicators of other components (step S04). Preferably, at least two statistical indicators are determined, and the comparison is made by indicator pair, an indicator pair of one component being compared to indicator pairs of other components. As a function of the comparison, the soundness or unsoundness of a component is determined (step S05), preferably on the basis of a deviation of the statistical indicators with respect to the others.

The comparison aims to identify the components for which the statistical indicator or indicators are remote from those of the sound components, assumed to be in the majority. Several approaches may be adopted. The comparison of the at least one indicator of said component to indicators of other components may comprise the determination of a difference between said statistical indicator and another nearer statistical indicator of another component, and even between said statistical indicator and several other nearer statistical indicators of other components. For example, the “K nearest neighbors” method consists, for each component, in computing the Euclidian distance to its K-th nearest neighbor. A threshold is then chosen for this distance to decide whether the component is potentially unsound. Other variants may be used, such as for example the “local outlier factor” method.

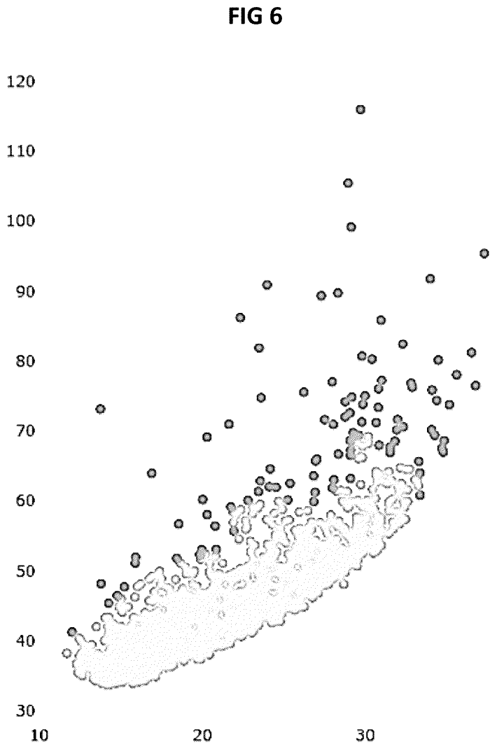

FIG. 6 shows an example of spatial representation in two dimensions of {mean, standard deviation} pairs for distances between baffle bolts. On the ordinate are the means and on the abscissae are the standard deviations. By applying the “K nearest neighbors” method, components whose statistical indicators deviated from the majority have been identified, and appear in black in FIG. 6, whereas the majority of the components whose statistical indicators were near one another appear in white, and which are therefore considered as sound.

Firstly, it was found that sound components have means and standard deviations greater than those of unsound components, and that certain unsound components have means well above those of the sound components, particularly greater than 70. Thus, a simple comparison of values to a fixed threshold would make it possible to detect certain unsound components, but would also determine that many unsound components are sound. Such an approach is therefore not completely satisfactory. Contrariwise, identifying the components whose indicators deviate from those of the majority of the others makes it possible to detect potentially unsound components even when they have lower means and standard deviations than the means and standard deviations of sound components.

Alternatively, the comparison of the indicator pair of one component to indicator pairs of other components comprises a statistical indicator of a probability density of the values taken by the pair of two statistical indicators of said component. One may for example use parametric methods (Gaussians, Gaussian mix) or else non-parametric methods, such as for example a kernel method.

Note that there is at least one statistical indicator per signal portion, such that the comparison can concern several statistical indicators of several signal portions of one and the same component. It is then for example possible to determine an unsoundness if it appears that the statistical indicator differs from the others for only one position in the sequence, or contrariwise require the statistical indicator to differ from the others in several positions in the sequence for the component to be considered unsound. In order not to miss any potentially defective components, it is preferable to consider as unsound an component whose statistical indicator or indicators differ from the others for at least one position in the sequence of signal portions.

Once the soundness or unsoundness of each of the components has been determined, it is possible to implement an in-depth inspection of the unsound components, or directly a physical intervention on them, such as their replacement or scrapping. In this respect, it should be noted that the method is configured to be capable of detecting all the unsound components, and it is less detrimental to consider a sound component as unsound rather than take the risk of deeming a component that is not sound as sound.

The invention is not limited to the embodiment described and shown in the appended figures. Modifications remain possible, particularly from the point of view of the composition of the various technical features or by substitution of technical equivalents, without however departing from the protective scope of the invention.

1. A method for non-destructive testing of a plurality of components based on measurement signals, each of the measurement signals corresponding to a predetermined different component, comprising the following steps:
obtaining a sequence of at least one signal portion based on each measurement signal, each of the measurement signals corresponding to a predetermined different component to be tested,
for each pair associating two signal portions having one and the same position in their respective sequence, a distance between these signal portions of said pair is computed, said distance being computed using dynamic time warping,
for each signal portion, determining at least one statistical indicator based on the distances of pairs involving said signal portion, the statistical indicator being associated with the component of said signal portion, and
for each component, comparing at least one indicator of said component to indicators of other components, and as a function of the comparison, determining the soundness or unsoundness of the component.

2. The method as claimed in claim 1, wherein two signal portions that follow one another in a sequence obtained from one and the same measurement signal partially overlap.

3. The method as claimed in claim 1, wherein the plurality of components comprises P components, P being a natural integer greater than 10, and each signal portion is involved in P-1 pairs.

4. The method as claimed in claim 1, wherein a statistical indicator is a central tendency indicator, such as the mean or the median.

5. The method as claimed in claim 1, wherein a statistical indicator is a dispersion indicator measuring the variability of the distances, such as the standard deviation or the variance.

6. The method as claimed in claim 1, wherein at least two statistical indicators are determined, comprising a central tendency indicator and a dispersion indicator.

7. The method as claimed in claim 1, wherein the comparison of at least one indicator of said component to indicators of other components comprises the determination of a difference between said statistical indicator and another nearer statistical indicator of another component.

8. The method as claimed in any of claims 1 to 7 claim 1, wherein at least two statistical indicators are determined, and the comparison of the indicator pair of one component to indicator pairs of other components comprises a statistical modelling of a probability density of the values taken by the pair of two statistical indicators of said component.

9. The method as claimed in claim 1, wherein each signal portion corresponds to a measurement distance at least equivalent to a typical size of sought defect.

10. A non-transitory computer readable medium comprising program code instructions stored thereon for causing a computer to execute the steps of the method as claimed in claim 1, when said medium is read by a computer.
